(19) United States
(12) Patent Application Publication
(10) Pub. No.: US 2022/0115140 A1
(43) Pub. Date: Apr. 14, 2022

(54) HEALTH SUPPORT APPARATUS, HEALTH SUPPORT METHOD, AND RECORDING MEDIUM RECORDING HEALTH SUPPORT PROGRAM

(71) Applicant: KABUSHIKI KAISHA TOSHIBA, Tokyo (JP)

(72) Inventors: Kenichi DONIWA, Asaka Saitama (JP); Takahiro TANAKA, Akishima Tokyo (JP); Kosuke HARUKI, Tachikawa Tokyo (JP); Taihei YAMAGUCHI, Chigasaki Kanagawa (JP)

(73) Assignee: KABUSHIKI KAISHA TOSHIBA, Tokyo (JP)

(21) Appl. No.: 17/460,782

(22) Filed: Aug. 30, 2021

(30) Foreign Application Priority Data
Oct. 13, 2020 (JP) 2020-172642

(51) Int. Cl. G16H 50/30; G16H 20/60; G16H 20/70; G16H 20/30

(52) U.S. Cl. CPC G16H 50/30; G16H 20/30; G16H 20/70; G16H 20/60

(57) ABSTRACT

A health support apparatus includes a processor including hardware. The processor accepts input of a first factor that is directly controlled by improving a lifestyle habit of a person subjected to a medical checkup and a second factor that is not directly controlled by improving a lifestyle habit of the person. The processor calculates change amounts of the first factor and the second factor. The processor predicts a disease risk value representing a disease risk of a disease of the person based on at least the change amount of the second factor.

### CROSS-REFERENCE TO RELATED APPLICATIONS

This application is based upon and claims the benefit of priority from the Japanese Patent Application No. 2020-172642, filed Oct. 13, 2020, the entire contents of which are incorporated herein by reference.

### FIELD

Embodiments described herein relate generally to a health support apparatus, a health support method, and a recording medium recording a health support program.

### BACKGROUND

Precision medicine has recently been proposed. The precision medicine is a medicine that analyzes medical treatment methods at the individual level and selects an optimal medical treatment method from the analyzed medical treatment methods. In health guidance against lifestyle-related diseases, optimal goal setting at the individual level is desired as is the case with precision medicine. For example, in order to reduce the diabetic risk, health guidance is desired to be provided in accordance with individual health conditions such that a given person is instructed to achieve 7% weight loss and another person is instructed to achieve 3% weight loss instead of uniformly instructing all persons to achieve 5% weight loss.

### BRIEF DESCRIPTION OP THE DRAWINGS

FIG. 1 is a block diagram showing the arrangement of an example of a health support apparatus according to the first embodiment;

FIG. 2 is a view showing an example of a lifestyle habit improvement pattern;

FIG. 3 is a block diagram showing the arrangement of an example of a factor change calculation unit;

FIG. 4 is a view showing an example of a weight change ratio table as an example of a first factor change table;

FIG. 5 is a view shewing an example of weight change ratio calculation results;

FIG. 6 is a view shewing an example of a second factor change table;

FIG. 7 is a block diagram showing an example of the hardware arrangement of the health support apparatus;

FIG. 8 is a flowchart showing the operation of the health support apparatus according to the first embodiment;

FIG. 9 is a block diagram showing the arrangement of an example of a health support apparatus according to the second embodiment;

FIG. 10 is a flowchart showing the operation of a health support apparatus according to the second embodiment;

FIG. 11A is view showing an example of a GUI for inputting Individual intention parameters;

FIG. 11B is a view showing another example of a GUI for inputting individual intentions;

FIG. 12 is a block diagram showing the arrangement of an example of a health support apparatus according to the third embodiment;

FIG. 13 is a flowchart showing the operation of the health support apparatus according to the third embodiment;

FIG. 14A is a block diagram showing the arrangement of a first example of a health support, apparatus according to the fourth embodiment;

FIG. 14B is a block diagram showing the arrangement or a second example or the health support apparatus according to the fourth embodiment; and

FIG. 15 is a flowchart showing the operation of the health support apparatus in the first example of the fourth embodiment.

### DETAILED DESCRIPTION

In general, according to one embodiment, a health support apparatus includes a processor including hardware. The processor accepts input of a first factor that is directly controlled by improving a lifestyle habit of a person subjected to a medical checkup and a second factor that is not directly controlled by improving a lifestyle habit of the person. The processor calculates change amounts of the first factor and the second factor. The processor predicts a disease risk value representing a disease risk of a disease of the person based on at least the change amount of the second factor.

The embodiments will be described below with reference to the accompanying drawings.

### First Embodiment

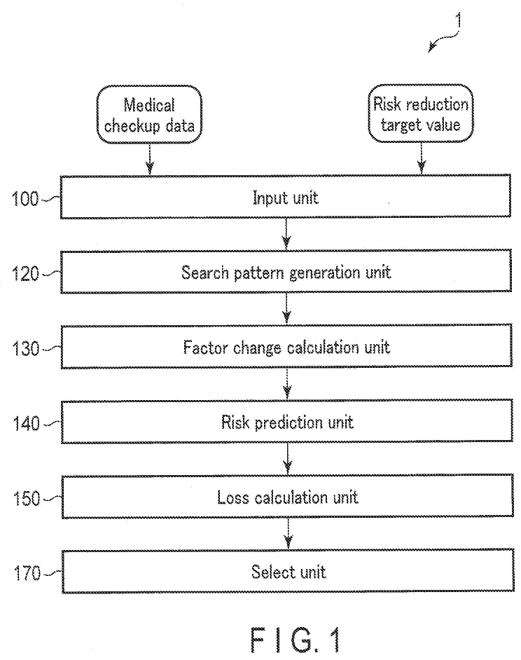

FIG. 1 is a block diagram showing the arrangement of an example of a health support apparatus according to the first embodiment. A health support apparatus 1 includes an input unit 100, a search pattern generation unit 120, a factor change calculation unit 130, a risk prediction unit 140, a loss calculation unit 150, and a select unit 170.

The input unit 100 accepts the input of medical checkup data and a risk reduction target value. For example, the input unit 100 may be configured to accept the input of medical checkup data and a reduction target value by the operation of the health support apparatus 1 by the user. In this case, the “user” is a person who uses the health support apparatus 1. The user may be a person subjected to a medical checkup, a doctor, or the like. In addition, the input unit 100 may be configured to accept medical checkup data stored in a storage medium outside the health support apparatus 1 (not shown) via a communication medium. Furthermore, the input unit 100 may be configured to accept medical checkup data transferred from the storage medium in response to when the storage medium is mounted in the health support apparatus 1.

In this case, medical checkup data is data recording a medical checkup result. Medical checkup data according to this embodiment includes the inspection value of a factor used for the prediction of the risk of a lifestyle-related disease and lifestyle habit information such as the amount of exercise habit, the amount of daily walking, and the frequency of alcohol drinking. Such factors include first factors and second factors. The first factors are factors that can be directly controlled by improving the lifestyle habit of a person subjected to a medical checkup. For example, the weight of the person is directly changed by executing exercise and improving the diet. Accordingly, the weight is included in the first factors. In addition, the body fat percentage and the like are included in the first factors. In contrast, the second factors are factors that cannot be directly controlled by improving the lifestyle habit of the person subjected to the medical checkup. For example, HbA1c (hemoglobin A1c) is not directly changed by executing exercise and improving the diet. Accordingly, HbA1c is included in the second factors. In addition, various biopsy values such as GOT (Glutamic Oxaloacetic Transaminase), LDL (Low Density Lipoprotein), and a blood pressure are included in the second factors. Note that HbA1c and the like are factors that cannot be directly controlled by improving the lifestyle habit but can be indirectly changed by a change in weight or the like accompanying the improvement of the lifestyle habit. That is, the second factors include factors that are indirectly controlled by changes in the first factors.

In addition, a risk reduction target value is a target value indicating to which degree the disease risk value of a specific disease such as a lifestyle-related disease is to be reduced. A risk reduction target value may be designated by a relative value or absolute value. In this case, the “disease risk value” is the onset probability of a specific disease in a given period. For example, when the disease risk of diabetes is to be reduced from 30% to 20%, the risk reduction target value (relative value) is 10 (%) when being designated by a relative value, whereas the risk reduction target value (absolute value) is 20 (%) when being designated by an absolute value.

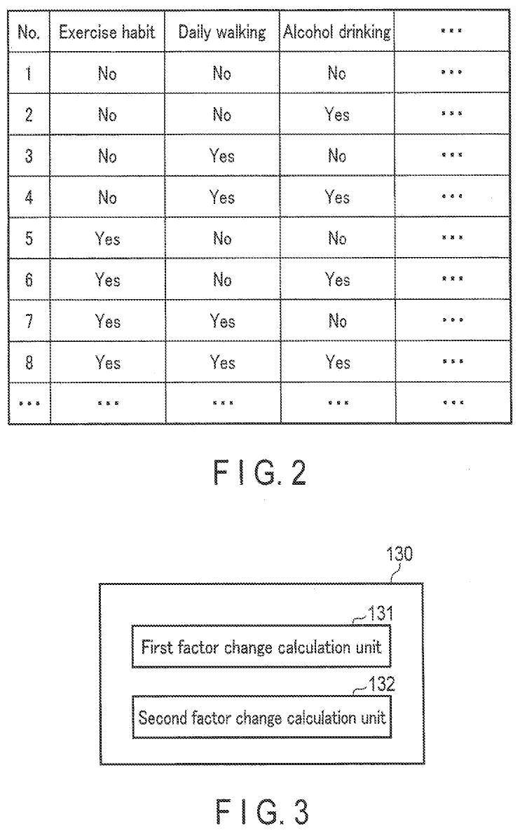

The search pattern generation unit 120 generates a lifestyle habit improvement search pattern. A lifestyle habit improvement search pattern includes a plurality of lifestyle habit combination patterns. FIG. 2 shows an example of lifestyle habit improvement combination patterns. The example shown in FIG. 2 indicates at least three items, namely “exercise habit”, “daily walking”, and “alcohol drinking” as lifestyle habits. The search pattern generation unit 120 generates a lifestyle habit improvement combination pattern by, for example, setting “yes” and “no” of these lifestyle habits. For example, setting “yes” for “exercise habit” and “daily walking” indicates that the lifestyle habits will be improved. On the other hand, setting “no” for “alcohol drinking” indicates that the lifestyle habit will be improved. For example, in pattern “No. 1”, “exercise habit”, “daily walking”, and “alcohol drinking” each are set to “no”. Pattern “No. 1” having such setting indicates that avoiding “alcohol drinking” will improve the lifestyle habits. In addition, in pattern “No. 2”, “exercise habit” and “daily walking” are set to “no”, and “alcohol drinking” is set to “yes”. Pattern “No. 2” having such setting indicates that, the lifestyle habits will not be improved concerning “exercise habit”, “daily walking”, and “alcohol drinking”. In this case, “no” in FIG. 2 does not indicate that “exercise habit”, “daily walking”, and “alcohol drinking” are not practiced at all. Note that “no” in FIG. 2 may include practicing “exercise habit”, “daily walking”, and “alcohol drinking” to such a degree that no influence is imposed on the prediction of a disease risk value. In addition, referring to FIG. 2, setting “yes” and “no” concerning the lifestyle habits will generate a lifestyle habit improvement pattern. In contrast to this, a lifestyle habit combination pattern may be generated in additional consideration of the amounts of the respective lifestyle habits. For example, “yes” for exercise habit may be divided into three patterns, namely, “little”, “intermediate”, and “much”.

In this case, the lifestyle habit data used for the generation of a lifestyle habit search pattern by the search pattern generation unit 120 is stored in, for example, the search pattern generation unit 120. Alternatively, the lifestyle habit data may be stored in a storage medium outside the health support apparatus 1. In this case, the search pattern generation unit 120 acquires lifestyle habit data from the external storage medium as needed.

The factor change calculation unit 130 calculates the change amounts of the first and second factors based on the lifestyle habit search pattern generated by the search pattern generation unit 120. For example, the factor change calculation unit 130 stores, in advance, a table including the change amount of factor for each lifestyle habit. The factor change calculation unit 130 calculates the change amounts of factors in accordance with the lifestyle habit search pattern generated by the search pattern generation unit 120. The factor change calculation unit 130 may totalize change ratios for the respective individual improvement patterns of lifestyle habits such as exercise and daily walking or may calculate a change amount by combining a plurality of lifestyle habits.

FIG. 3 shows the arrangement of an example of the factor change calculation unit 130. As shown in FIG. 3, the factor change calculation unit 130 may include a first factor change calculation unit 131 and a second factor change calculation unit 132. The first factor change calculation unit 131 calculates the change amount of a first factor based on the lifestyle habit search pattern. The second factor change calculation unit 132 calculates the change amount of a second factor. The second factor change calculation unit 132 calculates the change amount of a second factor based on the change amount of a first factor when a change in the first factor influences a change in the second factor.

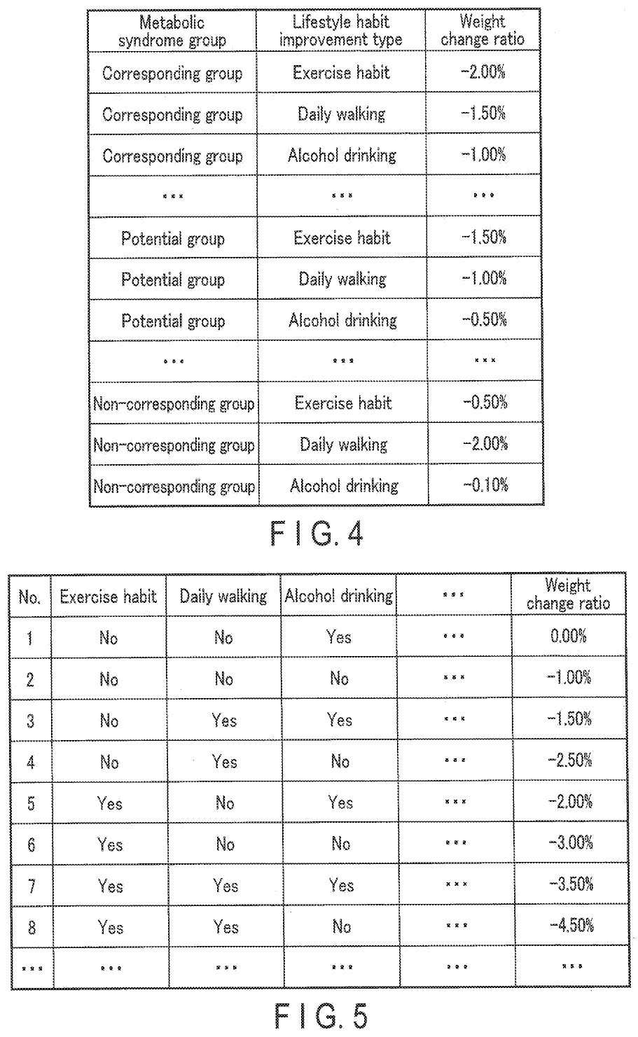

FIG. 4 shows an example of a weight change ratio table as an example of a first factor change table. The weight change ratio table is a table representing weight change ratios based on lifestyle habit improvements. A weight change ratio is a value representing a weight change by percentage. FIG. 4 shows an example in which weight change ratios are divided into three groups, namely a metabolic syndrome group, a potential metabolic syndrome group, and a non-metabolic syndrome group. A weight change ratio table can be generated by, for example, actually measuring weight changes accompanying lifestyle habit improvements concerning a large number o persons. The first factor change calculation unit 131 calculates a weight change ratio concerning a first factor by using the weight change ratio table and calculates the change amount of weight based on the calculated weight change ratio. The change amount of weight is, for example, the product of the weight of a person subjected to a medical checkup and a weight change ratio. In this case, the weight change ratio table may include weight change ratios corresponding to the amounts of lifestyle habits.

FIG. 5 shows an example of calculation results of weight change ratios. Referring to FIG. 5, a state of “no exercise habit”, “no daily walking habit”, and “alcohol drinking habit” is set as a reference state, and the weight change ratios shown are those, in the metabolic syndrome group, which are recorded when at least one of the lifestyle habits in the reference state is improved. For example, a lifestyle habit search pattern of “No. 2” in FIG. 5 is a pattern obtained by improving the alcohol drinking habit from “yes” to “no”. As shown in FIG. 4, the weight change ratio corresponding to “no alcohol drinking” in the metabolic syndrome group is −1.00%. Accordingly, the calculation result of the weight change ratio is −1.00%. Likewise, a lifestyle habit search pattern of “No. 4” in FIG. 5 is a pattern obtained by improving the daily walking habit from “no” to “yes” and the alcohol drinking habit from “yes” to “no”. As shown in FIG. 4, the weight change ratio corresponding to “yes for walking habit” in the metabolic syndrome group is −1.50%, and the weight change ratio corresponding to “no alcohol drinking” is −1.00%. Accordingly, the calculation result of the weight charge ratio is −2.50%.

In this case, FIG. 4 can be used as a table for the calculation of weight change ratios. A table similar to that shown in FIG. 4 is prepared for each type of first factors. The first factor change calculation unit 131 calculates the change amount of each first factor by using a corresponding first factor change table.

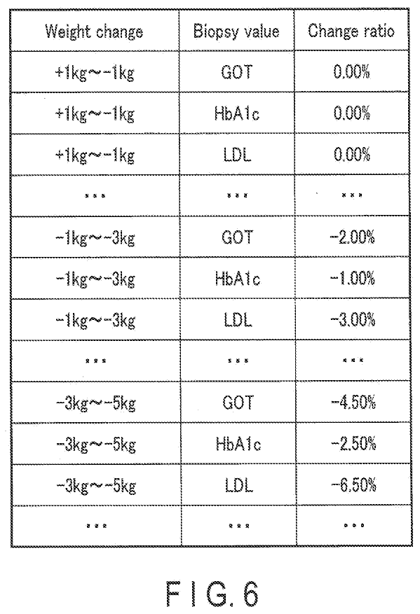

FIG. 6 shows an example of a second factor change table. FIG. 6 is a table representing the relation between weight change ratios and second factor change ratios. For example, Kazuyo Tsushita, “Practice and Evaluation of Lifestyle Intervention on Program”, The Journal of the Japanese Society of internal Medicine/Volume 105/Issue 9, [Online] [Search: Oct. 7, 2020], Internet URL: https://www.jstage.jst.go.jp/article/naika/105/9/105_1654 /_article/-char/ja/ reports that as the weight changes accompanying an improvement in lifestyle habit, a biopsy value such as GOT, HbA1c, or LDL as a second factor significantly changes. FIG. 6 shows the relation between such weight changes and changes in biopsy values as second factors. A second factor change table can be generated by, for example, actually measuring changes in biopsy values accompanying the weight changes of many persons. The second factor change calculation unit 132 calculates the change amounts of second factors based on the change amount of a first factor for each lifestyle habit search pattern calculated by the first factor change calculation unit 131. For example, when the weight change amount is from −1 kg to +1 kg, the change ratios of GOT, HbA1c, and LDL each are 0.00%. In contrast to this, when the weight change amount is from −1 kg to −3 kg, the change ratios of GOT, HbA1c, and LDL are respectively −2.00%, −1.00%, and −3.00%.

In this case, FIG. 6 can be used as a cable for the calculation of the change amounts of the second factors from weight change ratios. A table similar to that shown in FIG. 6 is prepared for each type of first factors. The second factor change calculation unit 132 calculates the change amount of each second factor by using a corresponding second factor change table.

The risk prediction unit 140 predicts the disease risk value of a disease for each search pattern. The risk prediction unit 140 predicts the disease risk value of the disease specified by a risk reduction target value based on, for example, a disease risk prediction model. This risk prediction model is a learning model configured to receive, for example, medical checkup data and output a disease risk value for each disease. In this case, medical checkup data includes first factors, second factors, and lifestyle habit information. As a disease risk prediction method, an arbitrary known prediction method can be used. As described above, a lifestyle habit is improved, a first factor such as weight and biopsy values as second factors such as GOT, HbA1c, and LDL change at given ratios. Accordingly, inputting lifestyle habit information and the values of first and second factors after changes accompanying an improvement in lifestyle habit will make the risk prediction model output a disease risk value that changes accompanying an improvement in lifestyle habit. Note that the risk prediction unit 140 is not limited to any specific arrangement as long as being configured to receive at least the value of a second factor and output a disease risk value for each disease. For example, the risk prediction unit 140 may be configured to output a disease risk value for each disease by using only first and second factors instead of using all medical checkup data.

The loss calculation unit 150 calculates a third loss Loss3 used for selection by the select unit 170 according to equation (1):

Loss3=Loss1+α×Loss2 (1)

where Loss1 is a first loss based on the difference between a disease risk value predicted by the risk prediction unit 140 and a risk reduction target value corresponding to the disease risk value, Loss2 is a second loss caused by changing a lifestyle habit used for prediction and each corresponding factor, that is, an improvement in lifestyle habit and a factor accompanying the improvement, and α is a parameter for adjusting the weights of the first Loss1 and the second loss Loss2.

The loss calculation unit 150 calculates the first loss Loss1 according to equations (2) given below:

a) If predicted disease risk value≤risk reduction target value, then

Loss1=0

b) If predicted disease risk, value>risk reduction target value, then

Loss1=(predicted disease risk value−risk reduction target value)² (2)

In addition, the loss calculation unit 150 calculates the second loss Loss2 according to equation (3) given below:

Loss2=Σ(Xi−Xi_org)²/Num_X (3)

where Xi is a candidate for the target value for the ith (i is a natural number) input value of a prediction model. In this case, the input of the prediction model, is medical checkup data and includes the first and second factors and lifestyle habit information. In addition, Xi_org is an actual inspection value of the ith input value of the prediction model. Furthermore, Num_X is the number of input values. Note that the respective input values can greatly differ in scale. In this case, the value of Loss2 tends to be influenced by an input value with a large scale. In order to suppress the influence of such a specific input value, the values of Xi and Xi_org may be standardized within the range from 0 to 1.

The calculation of losses by the loss calculation unit 150 according to equations (1) to (3) is an example. The loss calculation unit 150 may perform arbitrary loss calculation to calculate losses that can be used for the selection by the select unit 170.

The select unit 170 selects one or more lifestyle habit combination patterns by using the loss calculated by the loss calculation unit 150. For example, when a loss is calculated according to equation (1), a smaller value of the third loss Loss3 indicates that the disease risk value improved by the corresponding lifestyle habit combination pattern is closer to the risk reduction target value. Therefore, for example, when a loss is calculated according to equation (1), the select unit 170 selects one or more lifestyle habit combination patterns from generated search patterns in ascending order of the value of the third loss Loss3. Depending on the calculation of losses, a larger loss value may indicate that the disease risk value improved by the corresponding lifestyle habit combination pattern is closer to the risk reduction target value. In such a case, the select unit 170 selects one or more lifestyle habit combination patterns from generated search patterns in descending order of loss value.

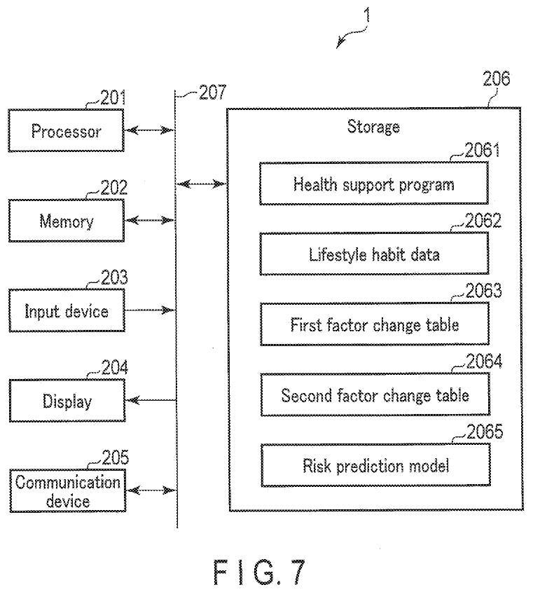

FIG. 7 shows an example of the hardware arrangement of the health support apparatus 1. The health support apparatus 1 includes, as hardware, for example, a processor 201, a memory 202, an input device 203, a display 204, a communication device 205, and a storage 206. The processor 201, the memory 202, the input device 203, the display 204, the communication device 205, and the storage 206 are connected to a bus 207. The health support apparatus 1 may be a terminal apparatus such as a personal computer (PC), smartphone, or tablet terminal.

The processor 201 is a processor that controls the comprehensive operation of the health support apparatus 1. The processor 201 operates as the input unit 100, the search pattern generation unit 120, the factor change calculation unit 130, the risk prediction unit 140, the loss calculation unit 150, and the select unit 170 by executing the health support program stored in, for example, the storage 206. The processor 201 is, for example, a CPU. The processor 201 may be an MPU, GPU, ASIC, FPGA, or the like. The processor 201 may be a single CPU or the like or a plurality of CPUs or the like.

The memory 202 includes a ROM and a RAM. The ROM is a nonvolatile memory. The ROM stores a boot program and the like for the health support apparatus 1. The RAM is a volatile memory. The RAM is used as a work memory when, for example, the processor 201 perform processing.

The input device 203 is an input device including a touch panel, a keyboard, and a mouse. When the input device 203 is operated, a signal corresponding to the operation is input to the processor 201 via the bus 207. The processor 201 performs various types of processing in accordance with such signals. The input device 203 can be used to input, for example, medical checkup data and a risk reduction target value.

The display 204 is a display such as a liquid display or organic EL display. The display 204 displays various types of images.

The communication device 205 is a communication device for allowing the health support apparatus 1 to communicate with an external device. The communication device 205 may be a communication device for wired communication or a communication device for wireless communication.

The storage 206 is a storage such as a flash memory, hard disk drive, or solid state drive. The storage 206 stores various types of programs executed by the processor 201, such as a health support program 2061. The storage 206 also stores lifestyle habit data 2062 for generating a lifestyle habit search pattern. The lifestyle habit data 2062 is, for example, an ID assigned to each lifestyle habit. The storage 206 also stores a first factor change table 2063 such as a weight change ratio table for the calculation of the change amount of a first factor. The storage 206 also stores a second factor change table 2064 such as a weight change ratio table for the calculation of the change amount of a second factor. The storage 206 also stores a risk prediction model 2065 used for the prediction of a disease risk value. The lifestyle habit data 2062, the first factor change table 2063, the second factor change table 2064, and the risk prediction model 2065 need not always be stored in the storage 206. For example, the lifestyle habit data 2062, the first factor change table 2063, the second factor change table 2064, and the risk prediction model 2065 may be stored in a server outside the health support apparatus 1. In this case, the health support apparatus 1 acquires necessary information by accessing the server by using the communication device 205.

The bus 207 is a data transfer path for exchanging data among the processor 201, the memory 202, the input device 203, the display 204, the communication device 205, and the storage 206.

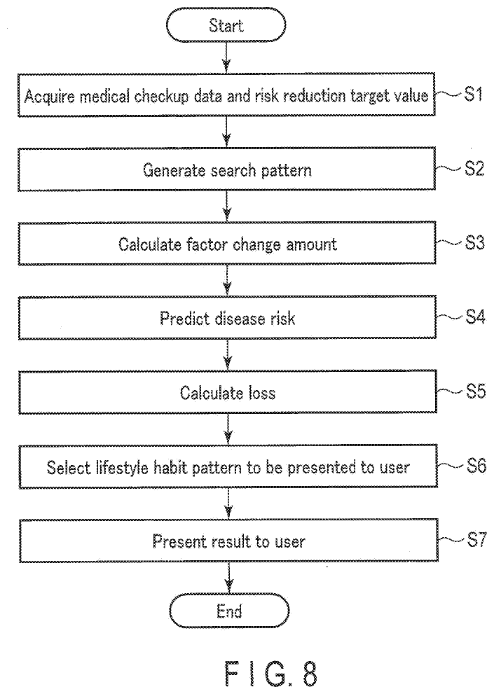

The operation of the health support apparatus 1 according to the first embodiment will be described next. FIG. 8 is a flowchart showing the operation of the health support apparatus 1 according to the first embodiment. The processing in FIG. 8 is executed by the processor 201.

In step S1, the processor 201 acquires medical checkup data and a risk reduction target value. The medical checkup data may be input via the operation of the input device 203 by the user on the GUI (Graphical User Interface) displayed on the display 204 or input via a storage medium outside the health support apparatus 1. The risk reduction target value may be input via the operation of the input device 203 by the user on the GUI displayed on the display 204.

In step S2, the processor 201 generates a lifestyle habit search pattern by referring to the lifestyle habit data 2062. The processor 201 may generate a search pattern based on all lifestyle habit combinations generated from the lifestyle habit data 2062 or generate a search pattern based on some lifestyle habit combinations.

In step S3, the processor 201 calculates the change amount of a factor used for the prediction of a disease risk for each search pattern, when a change in first factor influences a chance in second factor, the processor 201 calculates the change amount of the first factor first by using the first factor change table 2063 and then calculates the change amount of the second factor by using the second factor change table 2064.

In step S4, the processor 201 predicts a disease risk value. For example, the processor 201 predicts a disease risk value for each search pattern by inputting the value of the second factor after the change to a disease risk prediction model.

In step S5, the processor 201 executes loss calculation. For example, the processor 201 calculates losses according to equations (1) to (3).

In step S6, the processor 201 selects a lifestyle habit combination pattern to be presented to the user. For example, the processor 201 selects one or more lifestyle habit combination patterns in ascending order of the values of losses.

In step S7, the processor 201 presents the result to the user. Thereafter, the processor 201 terminates the processing in FIG. 8. For example, the processor 201 displays the selected lifestyle habit combination pattern and a disease risk value predicted from the corresponding lifestyle habits on the screen of the display 204. A reduction value of the disease risk value may be displayed instead of a disease risk value.

As described above, according to the first embodiment, a disease risk value is predicted by using the first factor that can be directly controlled by improving the lifestyle habit of a person subjected to a medical checkup and the second factor that cannot be directly controlled by improving the lifestyle habit of the person subjected to a medical checkup. In this case, when a change in second factor influences a change in first factor, the change in first factor is replaced with the change in second factor, and a disease risk value can be predicted based on the replaced second factor. For example, the weight is reduced over a long period of time, such as after half a year or one year, by improving a lifestyle habit, and the biopsy value is improved by a long-term reduction in weight. According to the first embodiment, a disease risk value can be calculated in consideration of such a long-term reduction in weight and a long-term change in biopsy value. In this manner, in the first embodiment, the second factor that cannot be directly controlled by improving the lifestyle habit of a person subjected to a medical checkup is properly reflected in a prediction result in the prediction of a disease risk value, and hence optimal goal setting at the individual level can be implemented in health guidance.

### Second Embodiment

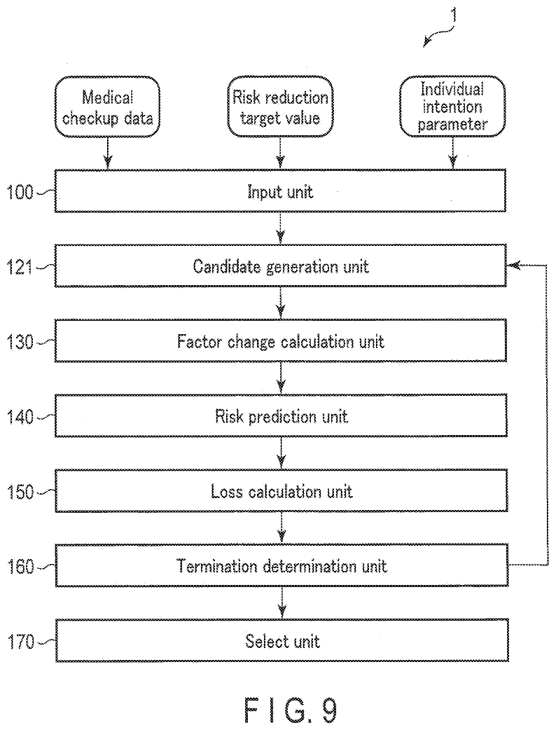

The second embodiment will be described next. FIG. 9 shows the arrangement of an example of a health support apparatus according to the second embodiment. A health support apparatus 1 according to the second embodiment includes an input unit 100, a candidate generation unit 121, a factor change calculation unit 130, a risk prediction unit 140, a loss calculation unit 150, a termination determination unit 160, and a select unit 170. A description of portions common to the first and second embodiments will be omitted or simplified. In addition, the arrangement shown in FIG. 7 can be applied to the hardware arrangement of the health support apparatus 1, and hence a description of the arrangement will be omitted.

The input unit 100 according to the second embodiment accepts the input of medical checkup data and a risk reduction target value. In addition, the input unit 100 according to the second embodiment accepts the input of individual intention parameters. An individual intention parameter is a parameter for reflecting, in changing a lifestyle habit to reduce a disease risk, an individual intention concerning an improvement in lifestyle habit for each person subjected to a medical checkup, such as an intention to mainly change a specific lifestyle habit or an intention not to change a specific lifestyle habit. For example, the input unit 100 may be configured to accept the input of an individual intention parameter by the operation of the health support apparatus 1 by the user.

The candidate generation unit 121 generates a lifestyle habit improvement candidate pattern in consideration of individual intention parameters. A lifestyle habit improvement candidate pattern is a predetermined lifestyle habit combination pattern. The candidate generation unit 121 generates a candidate pattern by setting “yes” or “no” for a lifestyle habit and, if “yes” is set, the corresponding amount. The candidate generation unit 121 may randomly generate a candidate pattern or generate a candidate pattern based on a Bayesian search (Bayesian optimization method) using the losses calculated by the loss calculation unit 150. That is, the candidate generation unit 121 may generate a lifestyle habit improvement candidate pattern by an arbitrary technique that can consider individual intention parameters.

The factor change calculation unit 130 calculates the change amounts of the first and second factors based on the candidate pattern generated by the candidate generation unit 121. The change amounts of the first and second factors may be calculated by the technique described in the first embodiment.

The risk prediction unit 140 predicts the disease risk value of a disease concerning the candidate pattern. The risk prediction unit 140 may perform prediction by the technique described in the first embodiment.

The loss calculation unit 150 calculates the third loss Loss3 according to, for example, equation (1) described above. In this case, in the second embodiment, the loss calculation unit 150 calculates the second loss Loss2 according to, for example, equation (4) given below.

Loss2=Σ(Xi−Xi_org)²×Xi_std/Num_X (4)

where Xi_std is a value corresponding to an individual intention parameter concerning the ith input value. Xi_std is a value in the range of, for example, 0 to 1. As Xi_std is reduced, the value of Loss2 decreases. For example, in Bayesian search, a next candidate pattern is generated so as to reduce the value of a loss, and hence a candidate pattern is generated so as to improve more a lifestyle habit corresponding to Xi_std that is reduced eventually. In contrast to this, as Xi_std is increased, the value of Loss2 increases. As a result, a candidate pattern is generated so as to improve less a lifestyle habit corresponding to Xi_std that is increased eventually. For example, when an individual intention parameter is set to reduce the disease risk by changing the exercise habit, Xi_std corresponding to the amount of exercise habit decreases, in addition, when an individual intention parameter is set to reduce the disease risk without changing the frequency of alcohol drinking, Xi_std corresponding to the frequency of alcohol drinking increases.

The termination determination unit 160 determines whether to terminate a search for a candidate pattern. If, for example, a predetermined number of searches, that is, the calculation of a third loss has been performed, the termination determination unit 160 determines to terminate the search. Alternatively, if the third loss has decreased below a predetermined threshold, the termination determination unit 160 may determine to terminate the search.

The select unit 170 selects one or more lifestyle habit combination patterns by using the loss calculated by the loss calculation unit 150. When, for example, a loss is calculated according to equation (1), the select unit 170 selects one or more lifestyle habit combination patterns from the generated candidate patterns in ascending order to the value of the third loss Loss3.

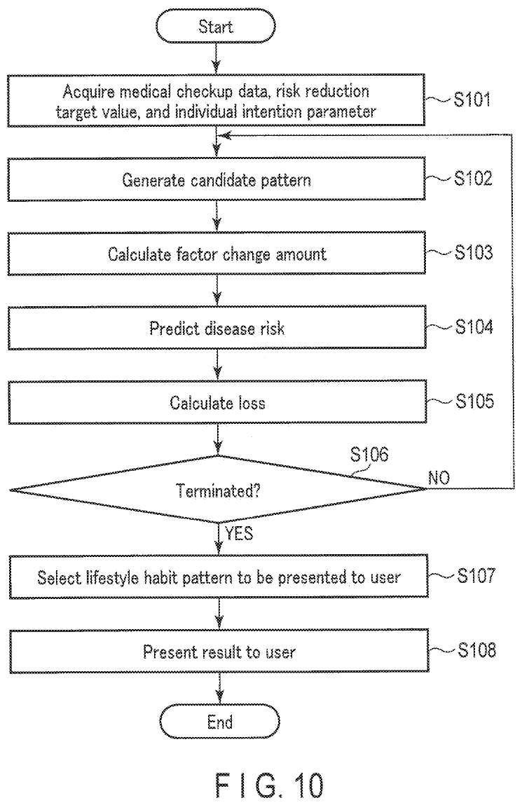

The operation of the health support apparatus 1 according to the second embodiment will be described next. FIG. 10 is a flowchart showing the operation of the health support apparatus 1 according to the second embodiment. The processing in FIG. 10 is executed by a processor 201.

In step S101, the processor 201 acquires medical checkup data, a risk reduction target value, and an individual intention parameter. The medical checkup data may be input, for example, via the operation of an input device 203 by the user on the GUI displayed on a display 204 or may be input via, for example, a storage medium outside the health support apparatus 1. The risk reduction target value may be input via the operation of the input device 203 by the user on the GUI displayed on the display 204. Furthermore, the individual intention parameter may be input via, for example, the operation of the input device 203 by the user on the GUI displayed on the display 204.

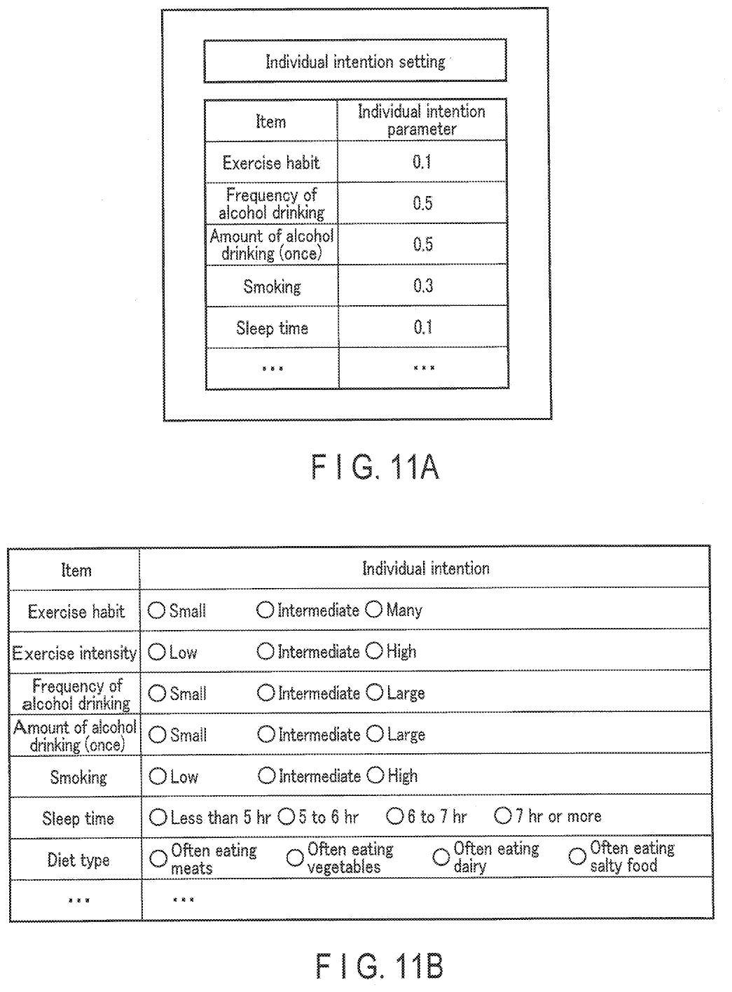

FIG. 11A shows an example of a GUI for inputting individual intention parameters. In the example in FIG. 11A, numerical values representing individual intention parameters are directly input. The user selects an individual intention parameter corresponding to a lifestyle habit which the user wants or does not want to change and inputs a numerical value in the range of, for example, 0 to 1. The value of Xi_std described above is set in accordance with the input numerical value. Referring to FIG. 11A, for example, “exercise habit”, “exercise intensity”, “alcohol drinking habit”, “alcohol drinking frequency”, “alcohol intake amount (once)”, “smoking”, “sleep time”, “diet type”, and the like are displayed as the respective items concerning lifestyle habits.

In step S102, the processor 201 generates a lifestyle habit candidate pattern by referring to lifestyle habit data 2062.

In step S103, the processor 201 calculates the change amount of a factor used for the prediction of a disease risk concerning the candidate pattern. When a change in first factor influences a change in second factor, the processor 201 calculates the change amount of the first factor first by using a first factor change table 2063 and then calculates the change amount of the second factor by using a second factor change table 2064.

In step S104, the processor 201 predicts a disease risk value. For example, the processor 201 inputs the value of the second factor after the change to a disease risk prediction model and predicts a disease risk value for each search pattern.

In step S105, the processor 201 executes loss calculation. For example, the processor 201 calculates losses according to equations (1), (2), and (4).

In step S106, the processor 201 determines whether to terminate the search. If the processor 201 does not determine in step S106 to terminate the search, the process returns to step S102. In this case, a next, candidate pattern is generated. If the processor 201 determines in step S106 to terminate the search, the process shifts to step S107.

In step S107, the processor 201 selects a lifestyle habit combination pattern presented to the user. For example, the processor 201 selects one or more lifestyle habit combination patterns in ascending order of the values of losses.

In step S108, the processor 201 presents the result to the user. The processor 201 then terminates the processing in FIG. 10. For example, the processor 201 displays the selected lifestyle habit combination pattern and the disease risk value predicted in the lifestyle habits on the screen of the display 204.

As described above, according to the second embodiment, a disease risk value is predicted in consideration of the individual intention of the person subjected to medical checkup in addition to the first and second factors. In the second embodiment, this makes it possible to make goal setting for lifestyle habits at the individual level mere properly than in the first embodiment.

In the second embodiment, individual intentions are input as numerical values of individual intention parameters. In contrast to this, the values of factors such as the amount of exercise habit and the frequency of alcohol drinking may be directly input, instead of the numerical values of individual intention parameters, to reflect the individual intentions. FIG. 11B shows another example of the GUI for inputting individual intentions. In another example, factors related to lifestyle habits and the options of the amounts of the factors are displayed. The user directly designates a lifestyle habit to be changed. For example, when changing the exercise habit to “much”, the user selects “much” in the exercise habit row in FIG. 11B. The value of Xi_org described above is associated with each option, and the value of Xi_org is set in accordance with the selection. When, for example, the exercise habit is changed to “much”, the value of Xi_org corresponding to the amount of exercise habit is fixed to “much”. Subsequently, the processor 201 executes a search while calculating the third loss in the same manner as in the first embodiment. With this operation, the frequency of the exercise habit is fixed to the state of “much”, and a search is executed by changing another lifestyle habit.

### Third Embodiment

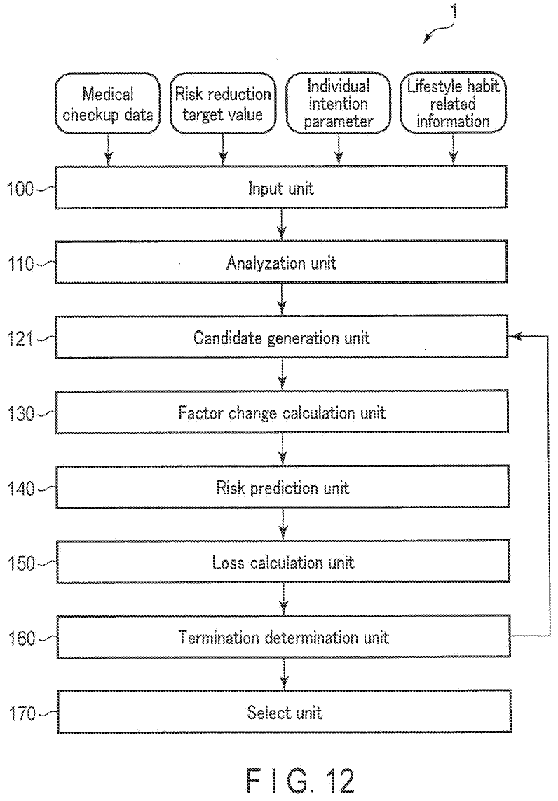

The third embodiment will be described next. FIG. 12 shows the arrangement of an example of a health support apparatus according to the third embodiment. A health support apparatus 1 according to the third embodiment includes an input unit 100, an analyzation unit 110, a candidate generation unit 121, a factor change calculation unit 130, a risk prediction unit 140, a loss calculation unit 150, a termination determination unit 160, and a select unit 170. In the third embodiment, a description of portions common to the second embodiment will be omitted or simplified. In addition, the arrangement shown in FIG. 7 can be applied to the hardware arrangement of the health support apparatus 1, and hence a description of the arrangement will be omitted.

The input unit 100 according to the third embodiment accepts the input of medical checkup data, a risk reduction target value, and an individual intention parameter. In addition, the input unit 100 according to the third embodiment accepts the input of lifestyle habit related information. Lifestyle habit related information is information related to the lifestyle habit of a person subjected to a medical checkup. For example, lifestyle habit related information is information related to the diet of a person subjected to a medical checkup. Information related to diet includes information such as a food purchase history and an eating-out history. Food purchase history information and eating-out history information can be specified by, for example, receipts, records of various types of point cards, and records of various types of prepaid cards. In addition, lifestyle habit related information includes, for example, the number of steps per day measured by a pedometer, the expenditure of calories, and PHR (Personal Health Record) managed for each person subjected to a medical checkup using a smartphone or the like.

The analyzation unit 110 analyzes lifestyle habit related information and adjusts an individual intention parameter based on the analyzation result. For example, the analyzation unit 110 can calculate the intake amount of alcohol from the purchase information recorded on a plurality of receipts. Upon confirming that the intake amount of alcohol is large, the analyzation unit 110 determines that the person subjected to a medical checkup has an alcohol drinking habit. It is known that while the intake amount of alcohol stays below a predetermined amount, the influence of alcohol drinking on health is small, whereas the intake amount exceeds the predetermined amount, the influence greatly increases. The analyzation unit 110 does not adjust any intention parameter while the intake amount of alcohol stays below the predetermined amount. However, when the intake amount of alcohol exceeds the predetermined amount, the analyzation unit 110 adjusts an individual intention parameter so as to reduce the frequency of alcohol drinking. In addition, for example, upon confirming that the number of steps per day is large, the analyzation unit 110 determines that the person has a walking habit. In this case, the analyzation unit 110 adjusts, for example, an individual intention parameter so as to reduce the disease risk while keeping the number of steps per day large.

The candidate generation unit 121, the factor change calculation unit 130, the risk prediction unit 140, the loss calculation unit 150, the termination determination unit 160, and the select unit 170 each may operate in the same manner as in the second embodiment. Accordingly, a description of each operation will be omitted.

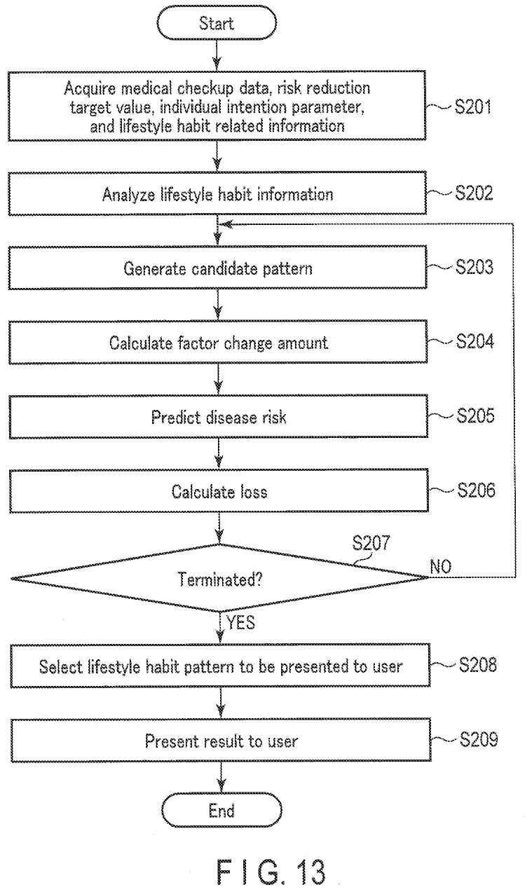

The operation of the health support apparatus 1 according to the third embodiment will be described next. FIG. 13 is a flowchart showing the operation of the health support apparatus 1 according to the third embodiment. The processing in FIG. 13 is executed by a processor 201.

In step S201, the processor 201 acquires medical checkup data, a risk reduction target value, an individual intention parameter, and lifestyle habit related information. The medical checkup data may be input, for example, via the operation of an input device 203 by the user on the GUI displayed on a display 204 or may be input via a storage medium outside the health support apparatus 1. The risk reduction target value may be input via, for example, the operation of the input device 203 by the user on the GUI displayed on the display 204. The individual intention parameter may be input via, for example, the operation of the input device 203 by the user on the GUI displayed on the display 204. The lifestyle habit related information may be input via, for example, the operation of the input device 203 by the user on the GUI displayed on the display 204, input from a terminal device such as a smartphone recording PHR, or input by recognizing characters from images of receipts or the like.

In step S202, the processor 201 analyzes lifestyle habit related information and adjusts the individual intention parameter based on the analyzation result.

In step S203, the processor 201 refers to lifestyle habit data 2062 and generates a lifestyle habit candidate pattern.

In step S204, the processor 201 calculates the change amount of a factor used for the prediction of a disease risk concerning the candidate pattern. When a change in first factor influences a change in second factor, the processor 201 calculates the change amount of the first factor first by using a first factor change table 2063 and then calculates the change amount of the second factor by using a second factor change table 2064.

In step S205, the processor 201 predicts a disease risk value. For example, the processor 201 predicts a disease risk value for each search pattern by inputting the value of the second factor after the change to the disease risk prediction model.

In step S206, the processor 201 executes loss calculation. For example, the processor 201 calculates losses according to equations (1), (2), and (4).

In step S207, the processor 201 determines whether to terminate the search. If the processor 201 does not determine in step S207 to terminate the search, the process returns to step S203. In this case, a next candidate pattern is generated. If the processor 201 determines in step S207 to terminate the search, the process shifts to step S208.

In step S208, the processor 201 selects a lifestyle habit combination pattern to be presented to the user. For example, the processor 201 selects one or more lifestyle habit combination patterns in ascending order of the values of losses.

In step S209, the processor 201 presents the result to the user. Subsequently, the processor 201 terminates the processing in FIG. 13. For example, the processor 201 displays the selected lifestyle habit combination pattern and the disease risk value predicted in the lifestyle habit on the screen of the display 204.

As described above, according to the third embodiment, an individual intention parameter is adjusted in accordance with lifestyle habit related information. With this operation, a disease risk value is predicted in consideration of the individual intention of the person subjected to a medical checkup even without any individual intention parameter input manually by the user. In the third embodiment, this makes it possible to make goal setting for lifestyle habits at the individual level more properly than in the second embodiment.

### Fourth Embodiment

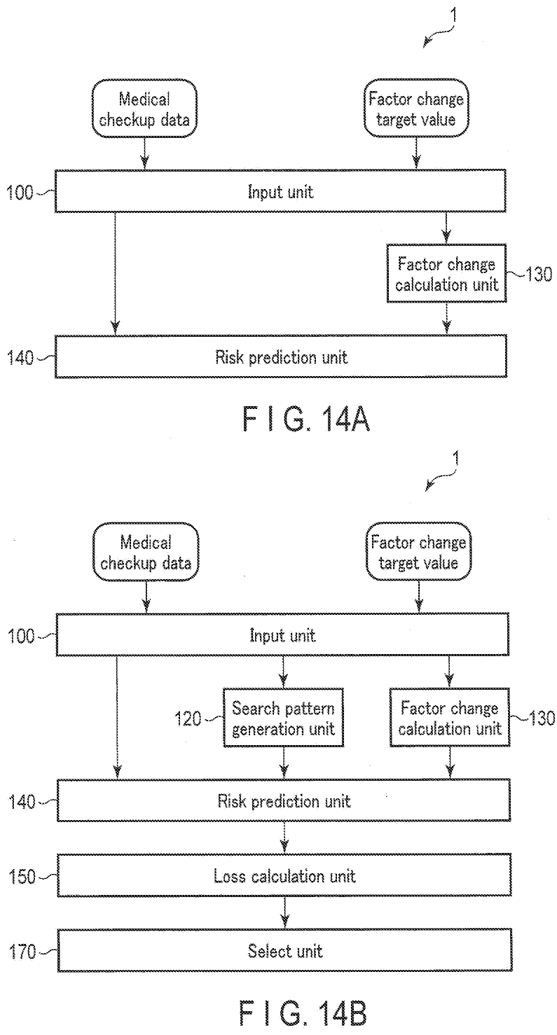

The fourth embodiment will be described next. FIG. 14A shows the arrangement of the first example of a health support apparatus according to the fourth embodiment. FIG. 14B shows the arrangement of the second example of the health support apparatus according to the fourth embodiment. In the first to third embodiments, a risk reduction target value is input, whereas in the fourth embodiment, a target value for the change of a factor is directly input.

The first example will be described first. A health support apparatus 1 according to the first example includes an input unit 100, a factor change calculation unit 130, and a risk prediction unit 140. A description of portions common to the first embodiment and the first example of the fourth embodiment, will be omitted or simplified. In addition, the arrangement shown in FIG. 7 can be applied to the hardware arrangement of the health support apparatus 1, and hence a description of the arrangement will be omitted.

The input unit 100 according to the first example of the fourth embodiment accepts the input of medical checkup data and a factor change target value. The factor change target value is a target value for the change of the factor. The factor change target value may be a target value for the change of a first factor such as weight or a target value for the change of a second factor such as blood pressure.

The factor change calculation unit 130 according to the first example of the fourth embodiment calculates the change amount of factor related to the change of the factor corresponding to an input factor change target value. Assume that a target value for the change amount of weight is input as a factor change target value. As described above, a change in weight also influences biopsy values such as GOT, HbA1c, and LDL. The factor change calculation unit 130 calculates the change amount of biopsy value such as GOT, HbA1c, or LDL from the change amount of weight. The change amount of factor can be calculated by using a factor change table similar to that in the first embodiment.

The risk prediction unit 140 predicts a disease risk value for each disease while the input factor change target value is set. The risk prediction unit 140 may perform prediction by using the technique described in the first embodiment.

The second example will be described next. The health support apparatus 1 according to the second example includes the input unit 100, a search pattern generation unit 120, the factor change calculation unit. 130, the risk prediction unit 140, a loss calculation unit 150, and a select unit 170. A description of portions common to the first embodiment and the second example of the fourth embodiment will be omitted or simplified. In addition, the arrangement shown in FIG. 7 can be applied to the hardware arrangement of the health support apparatus 1, and hence a description of the arrangement will be omitted.

The input unit 100 according to the second example of the fourth embodiment accepts the input of medical checkup data and a factor change target value as in the first example. The factor change target value may be a target value for the change of a first factor such as weight or a target value for the change of a second factor such as blood pressure.

The search pattern generation unit 120 generates a lifestyle habit improvement pattern. The lifestyle habit improvement pattern may be similar to that in the first embodiment.

The factor change calculation unit 130 according to the second example of the fourth embodiment calculates the change amounts of other first and second factors excluding the factor input as the factor change target value based on the lifestyle habit search pattern generated by the search pattern generation unit 120. The change amount of factor can be calculated by using a factor change table similar to that in the first embodiment.

The risk prediction unit 140 predicts a disease risk value for each disease while the input factor change target value is set. The risk prediction unit 140 may perform prediction by using the technique described in the first embodiment.

The loss calculation unit 150 calculates a third loss Loss3 according to, for example, equation (1) described above. Note, however, that in the second example of the fourth embodiment, the loss calculation unit 150 calculates a first loss Loss1 according to equation (5) given below:

Loss1=(F1−F2)² (5)

where F1 is the change ratio of a factor required to achieve the input factor change target value and F2 is the change ratio of a factor that can be achieved by improving a lifestyle habit. If, for example, an input factor change target value is a target value for GOT, F1 can be the change ratio of the weight required to achieve the GOT change target value and F2 can be the change ratio of the weight that, can be achieved by improving a lifestyle habit.

The termination determination unit 160 and the select unit 170 may operate in the same manner as in the first embodiment, and hence a description of the operations will be omitted.

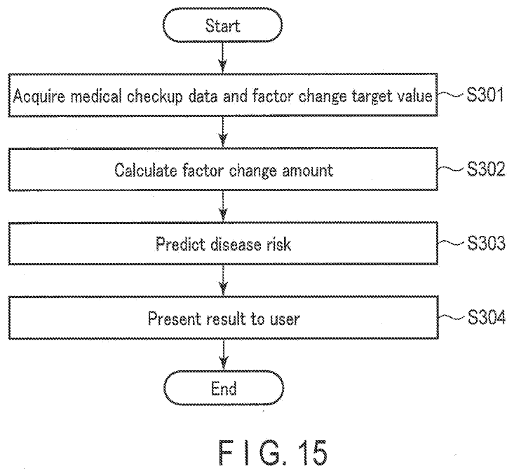

The operation of the health support apparatus 1 according to the fourth embodiment will be described next. FIG. 15 is a flowchart showing the operation of the health support apparatus 1 according to the first example of the fourth embodiment. The processing in FIG. 15 is executed by a processor 201. Note that the operation of the health support apparatus 1 according to the second example is the same as that in FIG. 15 except that the processing in steps S2, S3, S5, and S6 in FIG. 8 is added. Accordingly, a description of the operation will be omitted.

In step S301, the processor 201 acquires medical checkup data and a factor change target value. The medical checkup data may be input, for example, via the operation of an input device 203 by the user on the GUI (Graphical User Interface) displayed on a display 204 or may be input via a storage medium outside the health support apparatus 1. The factor change target value may be input via, for example, the operation of the input device 203 by the user on the GUI displayed on the display 204.

In step S302, the processor 201 calculates the change amount of a factor related to the input factor change target value. For example, when a weight change target value is input, the processor 201 calculates the change amount of biopsy value such as GOT, HbA1c, or LDL that changes with a change in weight.

In step S303, the processor 201 predicts a disease risk value. For example, the processor 201 predicts a disease risk value for each search pattern by inputting the value of a second factor after the change to the disease risk prediction model.

In step S304, the processor 201 presents the result to the user. Subsequently, the processor 201 terminates the processing in FIG. 15. For example, the processor 201 displays a disease risk value with respect to an input factor change target value on the screen of the display 204. A reduction value of the disease risk value may be displayed instead of a disease risk value.

As has been described above in the fourth embodiment, a target value for the change amount of a factor is input instead of a risk reduction target value, and a disease risk value is predicted by calculating the change amount of a factor related to the input target value. This also makes it possible to make goal setting for lifestyle habits at the individual level. In addition, in the fourth embodiment, a disease risk value can be predicted in accordance with a more direct lifestyle habit improvement target.

In the fourth embodiment, prediction may be performed in consideration of individual intention parameters as in the second and third embodiments. For example, replacing the search pattern generation unit 120 in the second example with a candidate generation unit 121 makes it possible to use the techniques according to the second and third embodiments without any change.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A health support apparatus comprising a processor including hardware and configured to
accept input of a first factor that is directly controlled by improving a lifestyle habit of a person subjected to a medical checkup and a second factor that is not directly controlled by improving a lifestyle habit of the person,
calculate change amounts of the first factor and the second factor, and
predict a disease risk value representing a disease risk of a disease of the person based on at least the change amount of the second factor.

2. The apparatus according to claim 1, wherein the second factor changes with a change in the first factor, and
the processor calculates a change amount of the first factor and calculates a change amount of the second factor based on the change amount of the first factor.

3. The apparatus according to claim 1, wherein the processor is configured to calculate change amounts of the first factor and the second factor for each lifestyle habit improvement combination pattern,
predict the disease risk value for each lifestyle habit improvement combination pattern based on at least the change amount, of the second factor,
generate the lifestyle habit improvement combination pattern, and
select not less than one of the lifestyle habit improvement combination patterns based on the predicted disease risk value and a reduction target value for the disease risk value.

4. The apparatus according to claim 3, wherein the processor is configured to calculate a first loss based on a difference between the predicted disease risk value and a reduction target value for the disease risk value, a second loss based on a difference between a lifestyle habit of the person and a target lifestyle habit of the person, and a third loss based on a sum of the first loss and the second loss, and
select not less than one lifestyle habit improvement combination pattern based on the third loss.

5. The apparatus according to claim 4, wherein the processor is configured to calculate the second loss further based on an individual intention parameter for reflecting an individual intention related to an improvement in the lifestyle habit of the person, and
generate a candidate for the lifestyle habit improvement combination pattern based on the third loss.

6. The apparatus according to claim 5, wherein the processor is configured to analyze lifestyle habit related information related to an input lifestyle habit of the person, and
adjust the individual intention parameter based on an analyzation result, on the lifestyle habit related information.

7. The apparatus according to claim 3, further comprising a display configured to display not less than one of the lifestyle habit improvement combination patterns.

8. The apparatus according to claim 1, wherein the first factor includes a weight of the person, and
the second factor includes a biopsy value of the person.

9. A health support apparatus comprising a processor including hardware and configured to
accept input of a first factor that is directly controlled by improving & lifestyle habit of a person subjected to a medical checkup, a second factor that is not directly controlled by improving a lifestyle habit of the person, and a reduction target value for at least one of the first factor and the second factor,
calculate change amounts of the first factor and the second factor excluding a factor corresponding to the reduction target value, and
predict a disease risk value representing a disease risk of a disease of the person based on at least the change amount of the second factor.

10. The apparatus according to claim 9, wherein the second factor changes with a change in the first factor, and
the processor calculates a change amount of the first factor and calculates a change amount of the second factor cased on the change amount of the first factor.

11. The apparatus according to claim 9, wherein the processor is configured to calculate change amounts of the first factor and the second factor for each lifestyle habit improvement combination pattern,
predict the disease risk value for each lifestyle habit improvement combination pattern based on at least the change amount of the second factor,
generate the lifestyle habit improvement combination pattern, and
select not less than one of the lifestyle habit improvement combination patterns based on the predicted disease risk value and a reduction target value for the disease risk value.

12. The apparatus according to claim 11, wherein the processor is configured to calculate a first loss based on a difference between the predicted disease risk value and a reduction target value for the disease risk value, a second loss based on a difference between a lifestyle habit of the person and a target lifestyle habit of the person, and a third loss based on a sum of the first loss and the second loss, and
select not less than one lifestyle habit improvement combination pattern based on the third loss.

13. The apparatus according to claim 12, wherein the processor is configured to calculate the second loss further based on an individual intention parameter for reflecting an individual intention related to an improvement in the lifestyle habit of the person, and
generate a candidate fox the lifestyle habit improvement combination pattern based on the third loss.

14. The apparatus according to claim 13, wherein the processor is configured to analyze lifestyle habit related information related to an input lifestyle habit of the person, and
adjust the individual intention parameter based on an analyzation result on the lifestyle habit related information.

15. The apparatus according to claim 11, further comprising a display configured to display not less than one of the lifestyle habit improvement combination patterns.

16. The apparatus according to claim 9, wherein the first factor includes a weight of the person, and
the second factor includes a biopsy value of the person.

17. A health support method comprising:
accepting input of a first factor that is directly controlled by improving a lifestyle habit of a person subjected to a medical checkup and a second factor that is not directly controlled by improving a lifestyle habit of the person;
calculating change amounts of the first factor and the second factor; and
predicting a disease risk value representing a disease risk of a disease of the person based on at least the change amount of the second factor.

18. A health support method comprising:
accepting input of a first factor that is directly controlled by improving a lifestyle habit of a person subjected to a medical checkup, a second factor that is not directly controlled by improving a lifestyle habit of the person, and a reduction target value for at least one of the first factor and the second factor;
calculating change amounts of the first factor and the second factor excluding a factor corresponding to the reduction target value; and
predicting a disease risk value representing a disease risk of a disease of the person based on at least the change amount of the second factor.

19. A computer-readable non-transitory recording medium recording a health support program for causing a computer to execute
accepting input of a first factor that is directly controlled by improving a lifestyle habit of a person subjected to a medical checkup and a second factor that is nor directly controlled by improving a lifestyle habit of the person,
calculating change amounts of the first factor and the second factor, and
predicting a disease risk value representing a disease risk of a disease of the person based on at least the change amount of the second factor.

20. A computer-readable non-transitory recording medium recording a health support program for causing a computer to execute
accepting input of a first factor that is directly controlled by improving s lifestyle habit of a person subjected to a medical checkup, a second factor that is not directly controlled by improving a lifestyle habit of the person, and a reduction target value for at least one of the first factor and the second factor,
calculating change amounts of the first factor and the second factor excluding a factor corresponding to the reduction target value, and
predicting a disease risk value representing a disease risk of a disease of the person based on at least the change amount of the second factor.
